(19) United States
(12) Patent Application Publication
(10) Pub. No.: US 2015/0157409 A1
(43) Pub. Date: Jun. 11, 2015

(54) MEDICAL MANIPULATOR AND MEDICAL IMAGING SYSTEM INCLUDING THE SAME

(71) Applicant: CANON KABUSHIKI KAISHA, Tokyo (JP)

(72) Inventor: Kazufumi Onuma, Kawasaki-shi (JP)

(21) Appl. No.: 14/407,441

(22) PCT Filed: Jun. 5, 2013
(86) PCT No.: PCT/JP2013/003530

§ 371 Date: Dec. 11, 2014

(30) Foreign Application Priority Data
Jun. 15, 2012 (JP) 2012-135454

(51) Int. Cl. A61B 19/00; A61B 5/00

(52) U.S. Cl. CPC A61B 19/2203; A61B 19/201; A61B 19/5244; A61B 5/0095; A61B 2019/5261; A61B 2019/5236; A61B 2019/5276; A61B 2019/5238; A61B 2019/5274

(57) ABSTRACT

The present invention relates to a medical manipulator 30 which includes: a driving unit 6 configured to at least include an vibration-type actuator 10; a manipulator portion 5 configured to at least include an inserting portion 18 to be inserted in a living body and configured to be connected to the driving unit 6 and be moved when driven by the driving unit 6; a support unit 4 configured to support the driving unit 6 and the manipulator portion 5; a driving circuit 9 configured to be connected to the vibration-type actuator 10 and output a driving signal 42 which drives a vibration-type actuator 10 to the vibration-type actuator 10; and a stress compensation unit 11 configured to reduce stress produced in the inserting portion 18 due to a motion of the living body.

### TECHNICAL FIELD

The present invention relates to a medical manipulator which includes a vibration-type actuator as a driving source. More particularly the present invention relates to a medical imaging system which includes the medical manipulator. cl BACKGROUND ART

With the advance of the robotics technique, there has been an increasing need to apply the robotics technique to a medical device. An exemplary driving source of a manipulator for which high control precision is requested includes a vibration-type actuator illustrated in FIG. 10. The vibration-type actuator may perform direct driving without using a reducer. Since the vibration-type actuator has the holding torque, it is possible to retain the posture of the manipulator even when not energized. Therefore, the vibration-type actuator is desirably applied to a medical manipulator for which highly precise position control is required.

PTL 1 discloses a tubular vibration-type actuator which is highly compatible with a nuclear magnetic resonance imaging (hereafter, “MRI”) device. The disclosed tubular vibration-type actuator includes a stator and a rotor disposed extending in a longitudinal direction of a tube as a driving source of a puncture device so as to face each other on either of an inner tube or an outer tube. In order to solve a problem of vibration of a medical manipulator caused by flexing vibration of the actuator due to a tubular arrangement, it is also disclosed to dispose either one of the stator or the rotor of the vibration-type actuator at either one of the other of a recessed portion or a projecting portion which are formed as spirals and are made to fit each other.

### CITATION LIST

### Patent Literature

PTL 1: Japanese Patent Laid-Open No. 2005-185072

### SUMMARY OF INVENTION

### Technical Problem

Since the vibration-type actuator has the holding torque, when the medical manipulator is in a stationary state, a support unit and the manipulator portion are locked with respect to the external force. If the subject is a living body, biological organs, such as a respiratory organ, a circulatory organ, a digestive organ, a sense organ and a muscular system, are displaced autonomously and continuously. It is difficult to control the displacement of these biological organs themselves.

Therefore, there has been a case in which, when relative positions of a biological organ and the medical manipulator are changed and unbalanced load is applied to the medical manipulator, whereby stress and distortion are caused in each part of the medical manipulator. The occurred stress and distortion may affect durability of the medical manipulator as the medical manipulator is operated repeatedly. Therefore, occurrence of stress and distortion has been a problem expected to be solved from viewpoint of reliability of the medical manipulator.

As described above, when the medical manipulator including a vibration-type actuator as the driving source is used inside the body of the subject, there has been a problem occurred due to the holding torque of the vibration-type actuator.

### Solution to Problem

The present invention provides a medical manipulator which includes: a driving unit configured to at least include a vibration-type actuator; a manipulator portion configured to at least include an inserting portion to be inserted in a living body and configured to be connected to the driving unit and be moved when driven by the driving unit; a support unit configured to support the driving unit and the manipulator portion; a driving circuit configured to be connected to the vibration-type actuator and output a driving signal which drives a vibration-type actuator to the vibration-type actuator; and a stress compensation unit configured to reduce stress produced in the inserting portion due to a motion of the living body.

### Advantageous Effects of Invention

According to the medical manipulator of the present invention, since a vibration-type actuator is provided, in addition to keeping the feature that highly precise direct driving is possible as a merit of unnecessity of a reducer, there are advantageous effects as follows to solve the problems regarding reliability described below. That is, the medical manipulator of the present invention may compensate for an amount of control of the vibration-type actuator so as to follow a motion of body tissue caused inside a body of a subject. This means that external force which the medical manipulator during a treatment support act receives may be reduced by controlling the vibration-type actuator to follow the motion of the living body and, as a result, the stress caused in the medical manipulator is reduced. Further, this means that it is possible to reduce a manipulator operation time related to the treatment support act by highly precisely controlling the medical manipulator. Therefore, in the medical manipulator of which target will move, it is possible to increase durability of the medical manipulator.

Further features of the present invention will become apparent from the following description of exemplary embodiments with reference to the attached drawings.

### BRIEF DESCRIPTION OF DRAWINGS

FIG. 1 is a block diagram illustrating an exemplary configuration of a first embodiment of a medical manipulator of the present invention which includes a displacement detecting unit.

FIG. 2A is an explanatory view illustrating a control mechanism using a medical image in the first embodiment of the medical manipulator of the present invention.

FIG. 2B is an explanatory view illustrating a control mechanism using a medical image in the first embodiment of the medical manipulator of the present invention.

FIG. 2C is an explanatory view illustrating a control mechanism using a medical image in the first embodiment of the medical manipulator of the present invention.

FIG. 3 is a block diagram illustrating a modification including a plurality of degrees of freedom as a first embodiment of the medical manipulator of the present invention.

FIG. 4A is a block diagram illustrating an exemplary configuration of a second embodiment of a medical manipulator of the present invention which includes a stress detecting unit.

FIG. 4B is a modification of the configuration illustrated in FIG. 4A.

FIG. 5A is an explanatory view illustrating a control mechanism in which a torque sensor is used in the second embodiment of the medical manipulator of the present invention.

FIG. 5B is a modification of the control mechanism illustrated in FIG. 5A.

FIG. 6 is a block diagram illustrating a modification which includes a plurality of degrees of freedom as a second embodiment of the medical manipulator of the present invention.

FIG. 7 is a block diagram illustrating a connecting relationship between the medical manipulator of the present invention and an external device.

FIG. 8A is a schematic configuration diagram illustrating a medical treatment support apparatus in which applied medical manipulator of the present invention has been applied to a medical puncture device.

FIG. 8B is a schematic configuration diagram illustrating a medical treatment support apparatus in which applied medical manipulator of the present invention has been applied to a medical biopsy device.

FIG. 9A is a diagram illustrating a connection condition between the medical manipulator of the present invention and a subject or a medical imaging device.

FIG. 9B is a diagram illustrating a connection condition between the medical manipulator of the present invention and a subject or a medical imaging device.

FIG. 9C is a diagram illustrating a connection condition between the medical manipulator of the present invention and a subject or a medical imaging device.

FIG. 9D is a diagram illustrating a connection condition between the medical manipulator of the present invention and a subject or a medical imaging device.

FIG. 10 is a schematic cross-sectional view illustrating an exemplary configuration of a vibration-type actuator which is applicable to the present invention.

### DESCRIPTION OF EMBODIMENTS

First, a basic structure of a vibration-type actuator 10 applicable to a medical manipulator of the present invention will be described with reference to FIG. 10. FIG. 10 is a dual cross sectional view illustrating a schematic structure of a ring-shape vibration-type actuator.

A ring-shape piezoelectric element 31 is bonded to a ring-shape vibrator 32. The piezoelectric element 31 excites vibration by an applied electrical signal. An exemplary electrical signal applied to the piezoelectric element 31 includes an alternating voltage signal. The vibrator 32 amplifies vibration excited by the piezo-electric element 31 as flexural vibration. Pressure is applied to between a movable body 2 and the vibrator 32 by pressurizing unit 3. Vibration by the vibrator 32 is transmitted to the movable body 2 by frictional force and the movable body 2 is rotated. The movable body 2 and a torque transmitting member 35 are joined by the pressurizing unit 3, and rotation of the movable body 2 is changed into rotation of an output shaft 36. The output shaft 36 is rotatable with respect to a housing 38 by a bearing 37. The vibrator 32 is fixed to the housing 38 by a connecting unit 39. In the present invention a structure consisting of the vibrator 32 and the piezoelectric element 31 is collectively referred to as a vibration body 1.

In the vibration-type actuator applicable to the medical manipulator of the present invention, the vibration body 1 and the movable body 2 are disposed in form of a ring around a rotation shaft 36.

Although the ring-shape configuration is described in the present embodiment, the present embodiment is not limited thereto. For example, the present invention includes various forms, such as a linear type in which the vibration body 1 and the movable body 2 are disposed linearly and a tubular type in which either one of the vibration body 1 or the movable body 2 is disposed on an inner tube or on an outer tube which constitute a double tube.

The pressurizing unit 3 applies pressure in the axial direction along the rotation shaft 36 but makes no deformation in the direction of rotation. For example, a plate spring may be used as the pressurizing unit 3. The vibration-type actuator has holding torque by axial direction pressure of the pressurizing unit 3. This is a point greatly different from that of an electromagnetism motor which uses Lorentz force as driving force. There is also an advantageous effect that, since the vibration-type actuator may operate at a lower speed and with larger torque as compared with the electromagnetism motor, it is easy to employ a direct drive mechanism excluding a reducer. Also in the present invention, it is a preferred form to cause the vibration-type actuator to perform a direct drive operation.

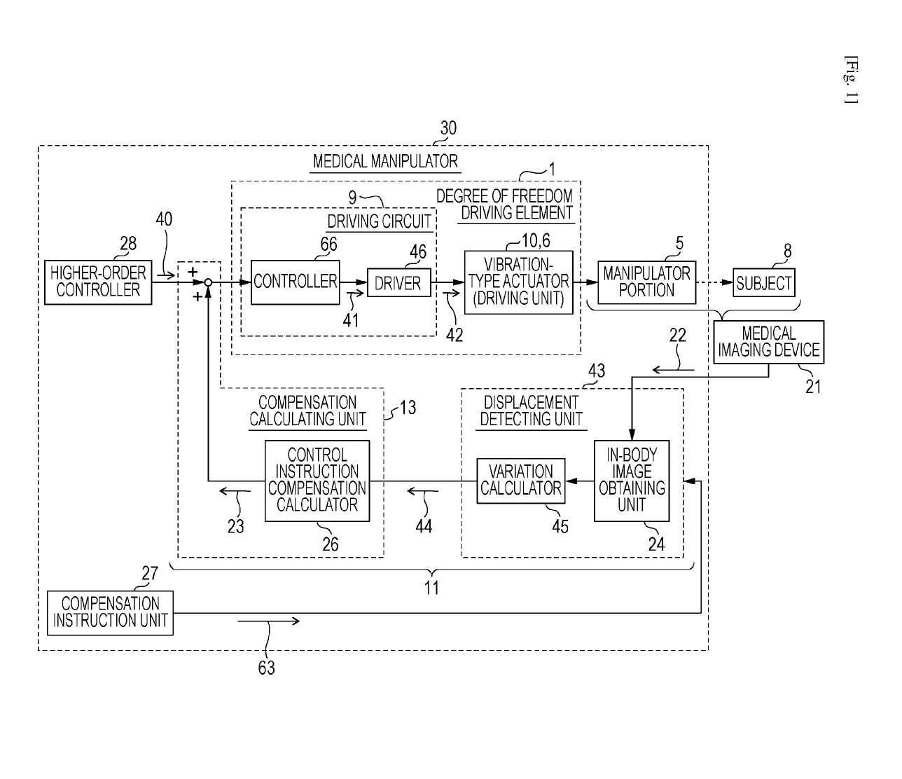

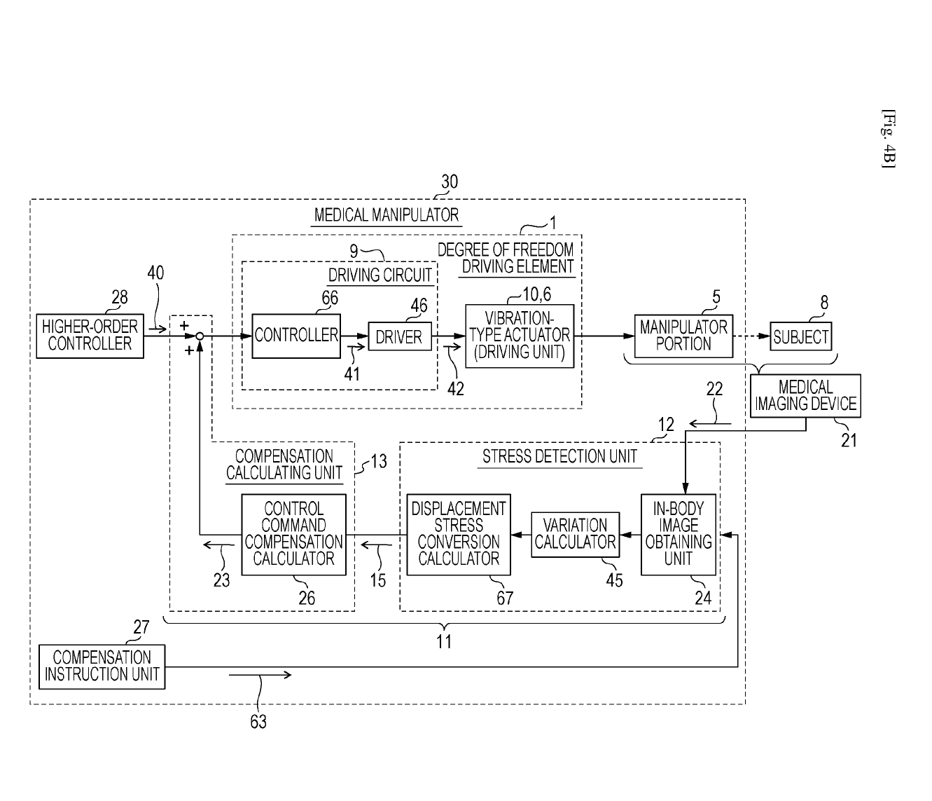

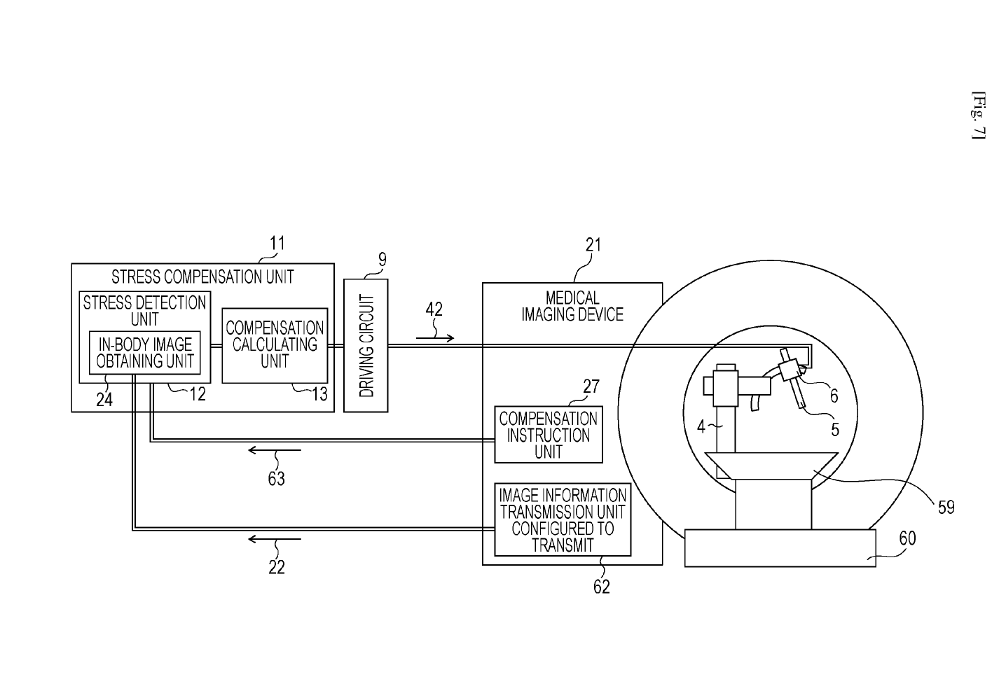

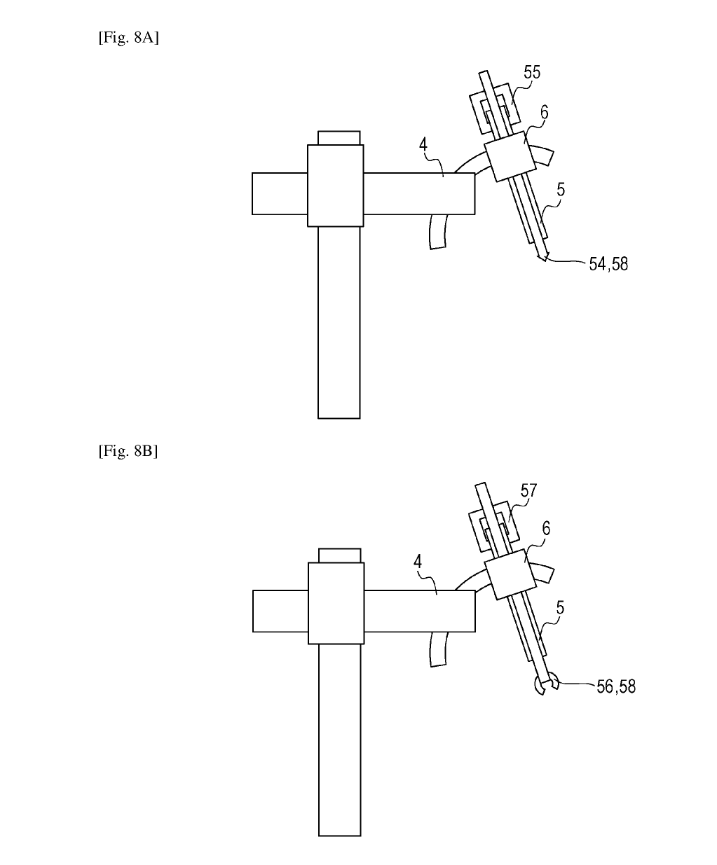

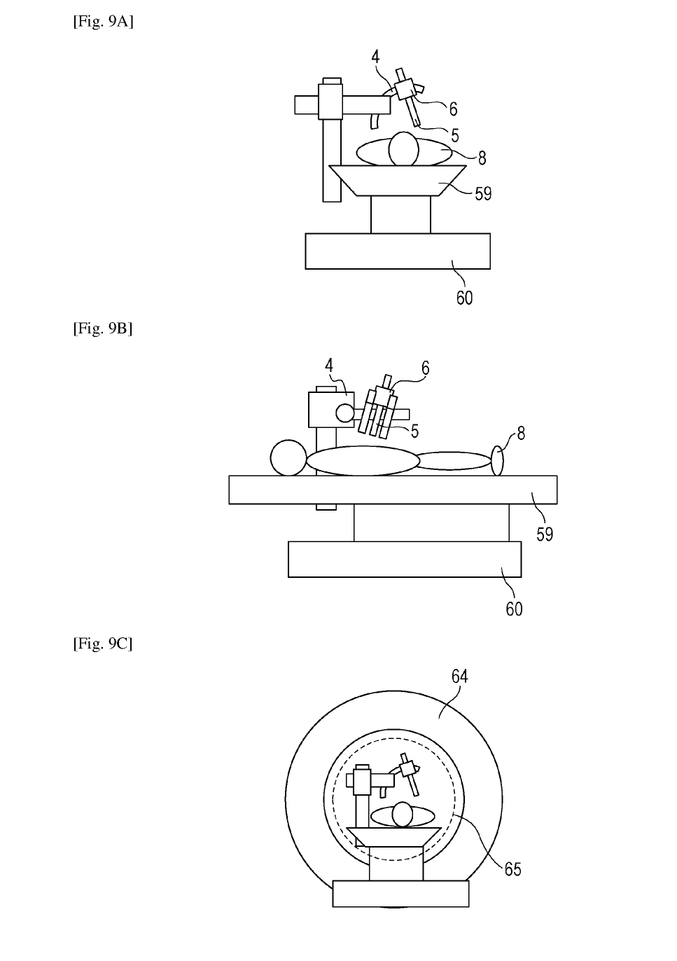

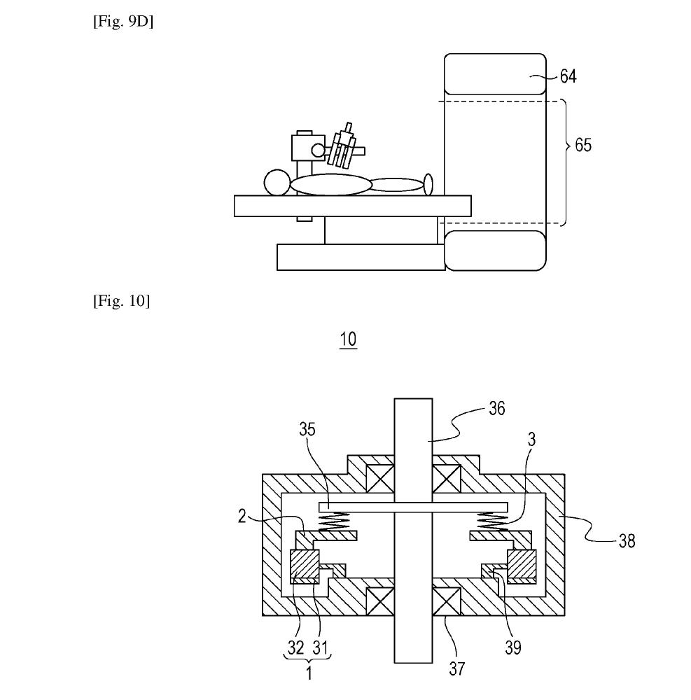

Next, a basic configuration of the present invention will be described with reference to FIGS. 1, 4A and 4B. As illustrated in each diagram of FIGS. 1, 4A and 4B, a medical manipulator 30 of the present invention includes a driving unit 6, a manipulator portion 5, a support unit (not illustrated), a driving circuit 9 and a stress compensation unit 11. The driving unit 6 at least includes the vibration-type actuator 10. The manipulator portion 5 at least includes an inserting portion (not illustrated) which is inserted in a living body. The manipulator portion 5 is connected to the driving unit 6 and is moved when driven by the driving unit 6. The support unit supports the driving unit 6 and the manipulator portion 5. The driving circuit 9 is connected to the vibration-type actuator 10 and outputs a driving signal 42 to the vibration-type actuator 10 for driving the vibration-type actuator 10. The stress compensation unit 11 reduces stress produced in an inserting portion 18 due to a motion of the living body. In the medical manipulator 30 of the present invention, there are at least two kinds of implementation forms of the stress compensation unit 11: one of them is that the medical manipulator 30 includes a displacement detecting unit 43 described to FIG. 1; and the other of them is that the medical manipulator 30 includes a stress detecting unit 12 illustrated in FIGS. 4A and 4B. For the convenience of illustration of the connection condition as a control system, the support unit and the inserting portion which are structural members are not illustrated in FIG. 1. The support unit and the inserting portion in the present invention will be described later with reference to FIGS. 8A to 9D. An exemplary embodiment of the present invention will be described with reference to FIGS. 1 to 7.

### First Embodiment

A first embodiment will be described with reference to FIGS. 1 to 3. FIG. 1 is a block diagram illustrating an extracted driving element of one degree of freedom for describing a control mechanism of the present embodiment. First, the present embodiment will be described with reference to FIG. 1.

FIG. 1 illustrates a basic structure used for a manipulate operation to a subject 8 in accordance with a control command 40 issued from a higher-order controller 28, and a stress compensation unit 11 which is a feature of the medical manipulator 30 of the present invention.

The basic structure includes, between the higher-order controller 28 and the subject 8, a driving circuit 9, the driving unit 6 which at least includes the vibration-type actuator 10, and the manipulator portion 5 which includes an unillustrated inserting portion which is moved when driven by the driving unit 6 and is inserted in the subject 8. The driving circuit 9 generates a driving signal 42 for driving the driving unit 6 in accordance with an input of a control command 40 as a higher-order instruction from a higher-order controller, and outputs the driving signal 42 to the driving unit 6. The driving circuit 9 may be constituted by a controller 66 and a driver 46. The controller 66 generates a control signal 41 in accordance with the control command 40. The driver 46 converts the control signal 41 into the driving signal 42.

FIG. 1 illustrates a medical imaging device 21 in which the manipulator portion 5 and the subject 8 are disposed within an imaging view. FIG. 1 also illustrates, as the stress compensation unit 11 which are features of the medical manipulator 30 of the present invention, a displacement detecting unit 43 connected to the medical imaging device 21 and a compensation calculating unit 13 connected to the displacement detecting unit 43. The compensation calculating unit 13 is further connected to a transmission path of the control command between the higher-order controller 28 and the driving circuit 9.

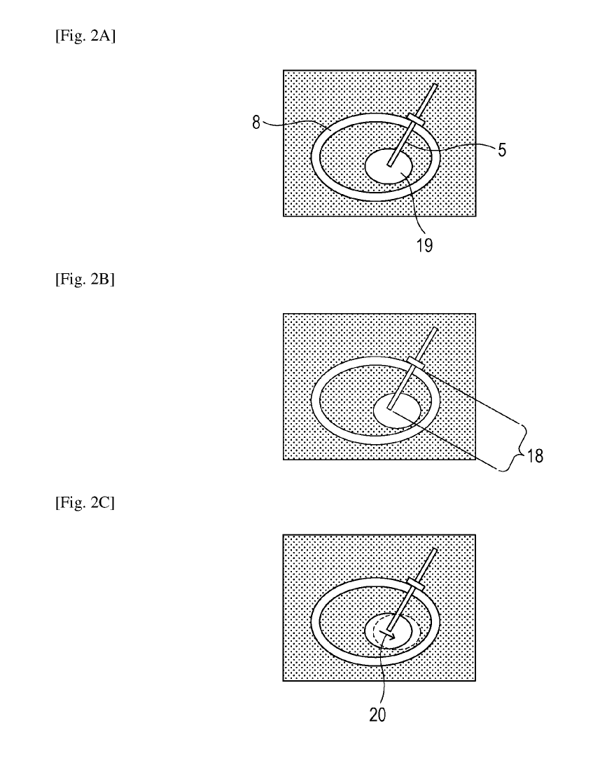

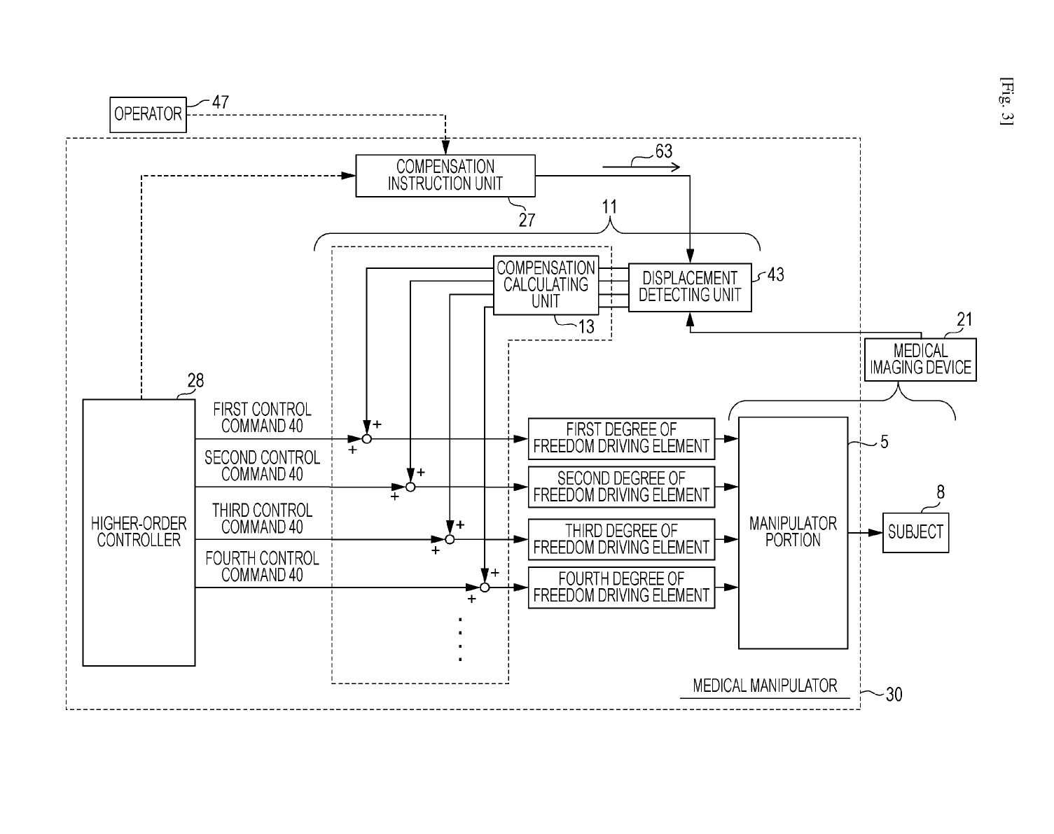

Next, the control mechanism of the present embodiment will be described with reference to FIGS. 1 to 2C.

In the first embodiment, including the stress compensation unit 11 consisting at least of the displacement detecting unit 43 and the compensation calculating unit 13 is a feature of the control mechanism. The stress compensation unit 11 adds a compensation signal as a calculation result of the compensation calculating unit 13 to a path from the higher-order controller 28 to the vibration-type actuator 10. With such a configuration, in the medical manipulator of the first embodiment, the inserting portion operates so that the stress received by the motion of the body of the subject may be reduced.

As illustrated in FIGS. 2A to 2C, when the inserting portion 18 is inserted inside the body of the subject 8, the inserting portion 18 receives external force due to a motion inside the body, whereby stress is produced in the inserting portion 18. However, in the present embodiment, as illustrated in FIG. 2C, it is not necessary to detect the stress produced in the inserting portion 18, in accordance with image information 22 obtained from the medical imaging device 21, the displacement detecting unit 43 detects fluctuation in a relative position between the inserting portion and a specific site of the subject as displacement information 44. Then, by adding a control command compensation signal 23 output from the compensation calculating unit 13 to the control command 40, a control operation which reduces stress produced in the inserting portion is implemented.

The compensation signal of the stress compensation unit 11 may be returned to an arbitrary point on a path from the higher-order controller 28 to the vibration-type actuator 10 as long as fluctuation of the relative displacement between the inserting portion and the specific site may be controlled. The compensation signal may be returned to, for example, the control command 40 as in the present embodiment, to the control signal 41 inside the driving circuit 9 or to the driving signal 42 as an input signal of the vibration-type actuator 10.

In the first embodiment, as illustrated in FIG. 1, the displacement detecting unit 43 may include an in-body image obtaining unit 24 which obtains image information 22 output from the medical imaging device. Though not particularly limited, the in-body image obtaining unit 24 is a unit which may obtain the image information 22 from the medical imaging device 21, such as an MRI device, an ultrasonic imaging device, a radiation imaging device and a photo-acoustic wave imaging device. By using such a medical imaging device 21, as illustrated in FIGS. 2A to 2C, image information which represents a positional relationship between a position of an internal organ of the subject and the treatment support instrument included in the inserting portion 18 or the manipulator portion 5 may be obtained.

Further, the displacement detecting unit 43 may include a displacement calculator 45 which generates fluctuation of a relative position between the inserting portion and the specific site as the displacement information 44 in accordance with the obtained image information 22 as illustrated in FIG. 1. The displacement calculator 45 obtains the fluctuation in the relative position between the internal organ and the inserting portion in accordance with the image information 22 obtained by the in-body image obtaining unit 24. In particular, the displacement calculator 45 obtains the fluctuation in the relative position in the following manner first, as illustrated in FIGS. 2A to 2C, an image which illustrates a reference positional relationship is obtained and then new images are obtained as needed. Each time a new image is obtained, the obtained image is compared with the reference image, fluctuation in position of the internal organ or the manipulator is obtained by image extraction. Then, a relative position fluctuation between the inserting portion and the specific site is obtained on the basis of the difference. In the present embodiment, the displacement detecting unit 43 has detected the relative displacement as detected displacement 20. However, in a case in which displacement of either of the manipulator portion 5 or the specific site of the subject is sufficiently small with respect to the another, only displacement of the another one may be detected and the displacement information 44 may be generated.

The displacement information 44 is output from the displacement detecting unit 43. Next, in accordance with the input of the displacement information 44, the compensation calculating unit 13 outputs the control command compensation signal 23. The control command compensation signal 23 is added to the control command 40 which is input to the controller 66. In this manner, since the fluctuation in the relative position between the manipulator portion 5 and the specific site of the subject is equivalent to the fluctuation in the target position, the vibration-type actuator 10 is driven by the controller 66 in the direction such that the fluctuation in the relative position becomes small. Thereby, the medical manipulator 30 in the present embodiment may synchronize the position of the treatment support instrument to the motion of, for example, the internal organ in the living body.

In the present invention, a state in which the stress compensation unit 11 is operated and the medical manipulator performs compensation control in synchronization with the motion of the specific site, such as an internal organ, is referred to as a compensation control state and a state in which no compensation control in synchronization with the motion of the specific site is performed but position control in accordance with an instruction of the higher-order controller is performed is referred to as a movement control state.

Next, an operation mechanism which switches the compensation control state and the movement control state will be described with reference to an example in which the medical manipulator of the present embodiment is applied to a treatment support act.

When the treatment support act is to be performed, it is necessary to precisely insert an unillustrated treatment support instrument in a target location in the living body. Therefore, at the time of insertion, position control is performed with the medical manipulator being put into the movement control state, and the manipulator portion 5 is moved so that the treatment support instrument is transported to a predetermined site in the living body.

Next, after the manipulator portion 5 arrives at the target point, it is necessary to keep the treatment support instrument inside the body for a predetermined period for performing the treatment support act. Then, in the present embodiment, when it is detected that the instrument has been inserted in the target point, the stress compensation unit 11 operates in accordance with the compensation instruction 63 output from the compensation instruction unit 27 and the state is changed from the movement control state to the compensation control state. Thereby, the manipulator portion is moved to follow the motion of the human body so as to reduce the stress produced in the inserting portion which is included in the manipulator portion. In this state, a treatment support act, such as a biopsy, is performed.

Next, when the treatment support instrument is to be drawn after the treatment support act, the compensation instruction unit 27 outputs an instruction for stopping compensation as the compensation instruction 63, and causes the state of the medical manipulator to be changed to the movement control state. Thereby, the medical manipulator is put in a state in which it will be driven to be controlled in accordance with the control command of the higher-order controller again, and the treatment support instrument is drawn by position control.

In the present embodiment, there may be some triggers with which the compensation instruction unit 27 issues the compensation instruction 63 to the displacement detecting unit 43. For example, if the stage is changed from the movement control state to the compensation control state after insertion, the compensation instruction unit 27 may automatically issue the compensation instruction 63 to the displacement detecting unit 43 with the completion of position control to the insertion target point as a trigger. Alternatively, determination of arriving at the target point and manipulation of a button and the like by an operator may be made as triggers.

As described above, when the medical manipulator of the first embodiment which includes the stress compensation unit 11 of the present invention is applied to the treatment support act, it is possible to reduce the stress produced in the inserting portion and to increase durability of the medical manipulator.

The higher-order controller 28 and the compensation instruction unit 27 may be included as components of the medical manipulator 30 as illustrated in FIG. 1A or may be provided as external components which issue commands to the medical manipulator 30 from the outside.

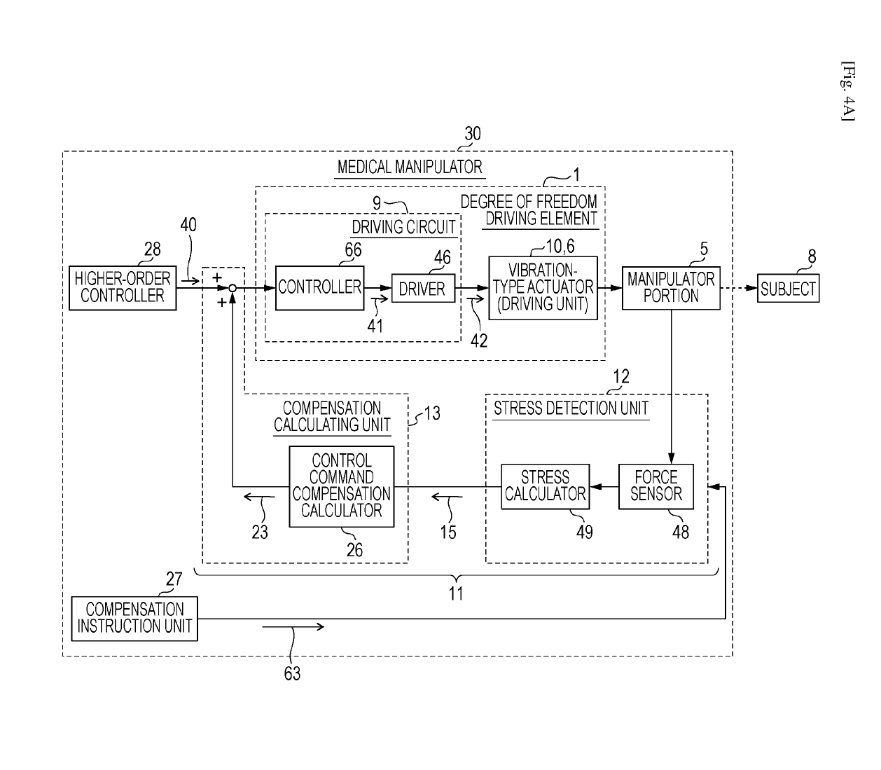

The medical manipulator 30 of the present invention may include a plurality of driving elements each having a different direction of control degree of freedom. In FIG. 1, for the ease of illustration, one degree of freedom is illustrated as an example. For example, a configuration including a plurality of control degrees of freedom as illustrated in the block diagram of FIG. 3 is also included in the first embodiment. In this case, as illustrated in FIG. 3, it is possible to output the control command compensation signal from the compensation calculating unit 13 independently to the driving element of each control degree of freedom. With this configuration, the compensation operation may be performed with respect to relative displacement corresponding to all the control degrees of freedom. However, it is not necessary to perform the compensation operation corresponding to all the control degrees of freedom. As needed, the control degree of freedom for performing compensation operation may be suitably selected or the compensation amount may be weighted between a plurality of control degrees of freedom. Exemplary selection of the control degree of freedom is setting the direction of the insertion shaft and the vertical direction of the manipulator portion 5 to be an axis which is the target of compensation operation and an axis which is not the target of the compensation operation, respectively.

In a medical manipulator which has a plurality of control degrees of freedom, the first embodiment is a desirable form in that position control of each of a plurality of control degrees of freedom may be performed with higher precision in accordance with image information by computed tomography from the medical imaging device 21.

### Second Embodiment

A second embodiment will be described with reference to FIGS. 4A to 6. FIG. 4A is a block diagram illustrating an extracted driving element of one degree of freedom for describing a control mechanism of the present embodiment. First, the present embodiment will be described with reference to FIG. 4A.

FIG. 4A illustrates a basic structure used for a manipulate operation to a subject 8 in accordance with a control command 40 issued from a higher-order controller 28, and a stress compensation unit 11 which is a feature of the medical manipulator 30 of the present invention.

The basic structure includes, between the higher-order controller 28 and the subject 8, a driving circuit 9, the driving unit 6 which at least includes the vibration-type actuator 10, and the manipulator portion 5 which includes an unillustrated inserting portion which is moved when driven by the driving unit 6 and is inserted in the subject 8. The driving circuit 9 generates a driving signal 42 for driving the driving unit 6 in accordance with an input of a control command 40 as a higher-order instruction from the higher-order controller 28, and outputs the driving signal 42 to the driving unit 6. The driving circuit 9 may be constituted by a controller 66 and a driver 46. The controller 66 generates a control signal 41 in accordance with the control command 40. The driver 46 converts the control signal 41 into the driving signal 42.

FIG. 4A also illustrates, as a stress compensation unit 11 which are features of the medical manipulator 30 of the present invention, a stress detecting unit 12 connected to a manipulator portion 5 and a compensation calculating unit 13 connected to the stress detecting unit 12. The compensation calculating unit 13 is further connected to a transmission path of the control command 40 between the higher-order controller 28 and the driving circuit 9.

The higher-order controller 28 and the compensation instruction unit 27 may be included as components of the medical manipulator 30 in the same manner as in the first embodiment, or may be provided as external components which issue commands to the medical manipulator 30 from the outside.

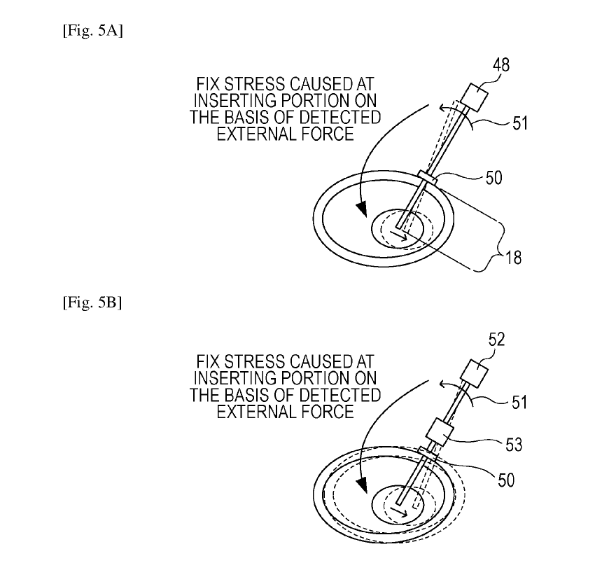

Next, the control mechanism of the present embodiment will be described with reference to FIGS. 4A and 5.

In the second embodiment, the control mechanism includes the stress compensation unit 11 which at least includes the stress detecting unit 12 and the compensation calculating unit 13. The stress compensation unit 11 adds a compensation signal obtained as a calculation result of the compensation calculating unit 13 to a path from the higher-order controller 28 to an vibration-type actuator 10. With such a configuration, in the medical manipulator 30 of the second embodiment, the manipulator portion 5 which includes the inserting portion operates so that the stress caused by the motion of the body of the subject 8 is reduced.

In the present embodiment, as illustrated in FIGS. 5A and 5B, the stress detecting unit 12 detects the stress received by the inserting portion 18 as stress information 15 at a force sensor 48 fixed to the manipulator portion 5 and at the stress calculator 49. Then, by adding a control command compensation signal 23 output as a calculation result of the compensation calculating unit 13 to the control command 40, a control operation which reduces stress which the inserting portion receives is implemented.

The stress detecting unit 12 in the present embodiment may include a force sensor 48 and a stress calculator 49. The force sensor 48 may be formed by a force sensor which detects force in a plurality of directions, or a load sensor which includes a strain gauge load cell. The force sensor 48 is fixed at least to any one of the manipulator portion 5, the driving unit 6 and the support unit 4. The force sensor 48 detects external force applied to the medical manipulator 30 and outputs external force information to the stress calculating unit 49. As illustrated in FIG. 5A, in accordance with the size and the direction of the force detected by the force sensor, the stress calculating unit 49 calculates the external force applied to the inserting portion 18 due to the motion of the subject 8 and the stress caused by the external force, and generates stress information 15. In FIG. 5A, since the inserting point 50 serves as a fulcrum, and the structure and the dimension of the manipulator portion 5 are already known, the stress of the inserting portion 18 produced in the direction opposite to that of the force detected by the force sensor 48 may be calculated and identified.

The fixed position of the force sensor is not particularly limited. Desirably, the fixed position is closer to a position at which the external force occurs, such as the treatment support instrument and a holding portion of the treatment support instrument. As illustrated in FIG. 5B, since a plurality of force sensors are provided, the stress applied to the inserting portion 18 may be detected highly precisely.

The compensation signal of the stress compensation unit 11 may be returned to, in the same manner as in the first embodiment, an arbitrary point on a path from the higher-order controller 28 to the vibration-type actuator 10 as long as stress caused in the inserting portion 18 due to the motion of the subject at a specific site is controllable.

Next, a modification of the second embodiment will be described with reference to FIG. 4B. FIG. 4B illustrates a configuration in which an in-body image obtaining unit 24 and a displacement calculating unit 45 are provided as the stress detecting unit 12 in the same manner as in the first embodiment. In the present embodiment, the stress detecting unit 12 further includes a displacement stress conversion calculator 67, whereby it is possible to detect the stress information 15 from the image information 22. Thereby, the medical manipulator of the present embodiment may reduce the stress in the inserting portion caused by the motion inside of the body of the subject.

It is possible to suitably switch the state of the medical manipulator according to the second embodiment of the present invention between a compensation control state and a movement control state in accordance with a compensation instruction 63 output from the compensation instruction unit 27. This feature is the same as that of the first embodiment. Therefore, it is possible to reduce the stress at the inserting portion and to increase the durability of the medical manipulator by applying the medical manipulator of the second embodiment to a treatment support act.

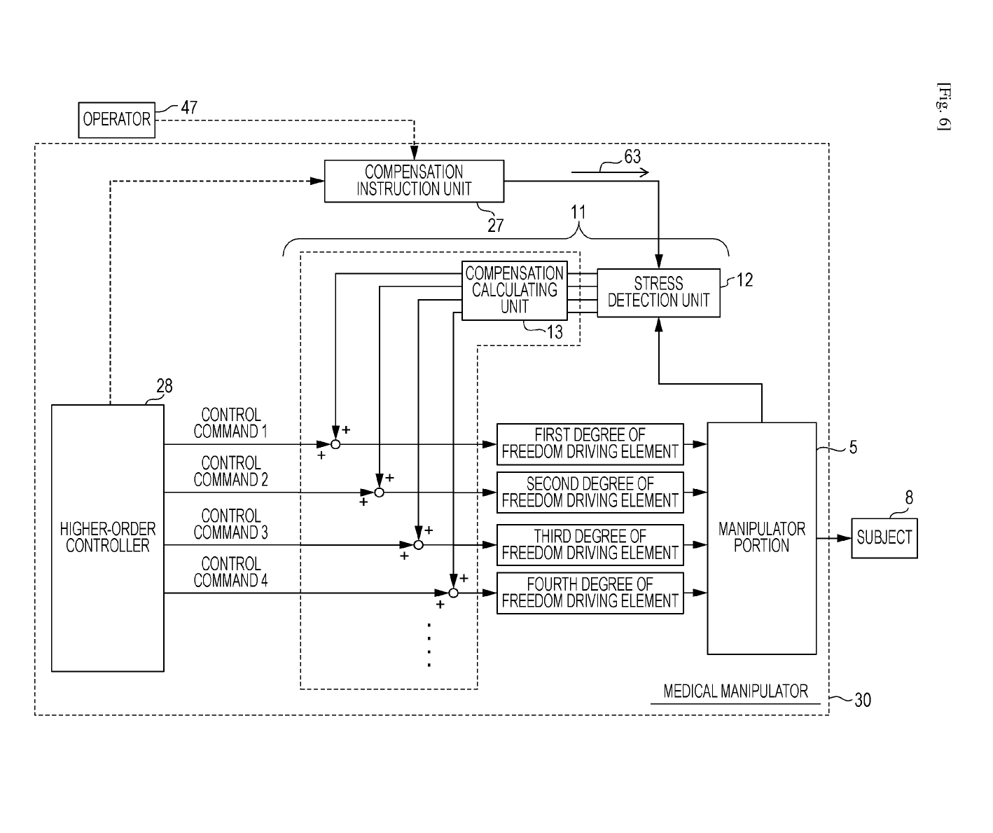

Although the driving element having one degree of freedom as illustrated in FIG. 4A has been described in the present embodiment, the driving element is not particularly limited to the same. The driving element may have a plurality of control degrees of freedom as illustrated in a block diagram of FIG. 6 in the same manner as in the first embodiment.

In the present embodiment, fluctuation in external force applied to the inserting portion is detected using the force sensor of the stress detecting unit 12 and the detected fluctuation is added to the control command 40 of the controller 66. If the control command 40 from the higher-order controller 28 and the controller 66 take the form of a torque reference, it is possible to cause the control command 40 between the higher-order controller 28 and the controller 66 to return the compensation signal without any complicated computation process performed by the compensation calculating unit 13 to the stress information 15.

In the present embodiment, external force is detected using the force sensor. Therefore, in a state in which the manipulator portion is outside the body, it is possible to reduce the decrease of the durability of the medical manipulator with respect to unavoidable shock and stress load to the manipulator portion due to, for example, a misoperation.

According to the medical manipulator of the present invention, the manipulator may be operated also manually because passivity is demonstrated in the direction in which the stress is reduced with respect to the target which is brought into contact with the manipulator portion 5 and the stress is caused at a predetermined timing based on the instruction from the compensation instruction unit 27. Therefore, usability at the time of installation and the like is improved.

Since the present embodiment has the above-described configuration, even if the subject which is the target moves, highly precise driving and the passive operation of the manipulator in which the actuator having high stationary torque is used may be switched. Therefore, stress applied to the medical manipulator of which target moves may be reduced and the durability of the medical manipulator may be increased.

In any of the embodiments, the driving unit 6 at least includes the vibration-type actuator 10 as a driving source for causing displacement of the manipulator portion 5 relative to the support unit 8. As needed, for example, the driving unit 6 may include a mechanical transmission member or an electromagnetic clutch. Therefore, in the medical manipulator of the present invention, it may be considered that the vibration-type actuator 10 is a member which constitutes the driving unit 6.

FIG. 7 illustrates a medical imaging system in which the medical manipulators of the first and the second embodiments of the present invention illustrated in the block diagrams of FIG. 1 and FIG. 4B, respectively, are connected to a medical imaging device 21 which performs imaging of in-body information of the subject. The medical imaging device 21 illustrated in FIG. 7 is an MRI device. The medical imaging device 21 includes the compensation instruction unit 27. In accordance with an analysis result of a computed tomography image, the compensation instruction unit 27 outputs the compensation instruction 63 to the stress compensation unit 11 of the medical manipulator of the present invention. Further, FIG. 7 illustrates a stress compensation unit 11 which includes an in-body image obtaining unit 24 in which input image information 22 output from the image information transmission unit 62 included in the medical imaging device 21 may be input. In FIG. 7, the medical imaging device 21 may also include an unillustrated higher-order controller.

### Other Embodiments

Next, a modification in which the manipulator portion 5 includes a treatment support instrument 58 will be described with reference to FIGS. 8A and 8B. In FIGS. 8A and 8B, the treatment support instrument 58 corresponds at least to the inserting portion inserted inside the body of a subject.

In FIG. 8A, a puncture unit 54 which is made to puncture inside the body of the subject is connected so that relative displacement is possible with respect to the manipulator portion 5. One end of the puncture unit 54 is fixed to a puncture unit driving unit 55 and the puncture unit driving unit 55 is fixed to the manipulator portion 5. With such a connecting relationship, relative position control of the puncture unit 54 with respect to the manipulator portion 5 is possible and relative position control of the puncture unit 54 with respect to the support unit 4 is possible. Since the medical manipulator of the present invention has such a configuration, it is possible to increase the durability of highly efficient medical manipulator which supports a puncture treatment act.

FIG. 8B illustrates a modification of the medical manipulator of the present invention in which a support unit 56 configured to obtain body tissue of the subject and an obtain unit driving unit 57 are disposed instead of the puncture unit 54 and the puncture unit driving unit 55 illustrated in FIG. 8A, respectively, so as to support a biopsy treatment act.

In FIGS. 8A and 8B, the stress compensation unit which is the feature of the present invention, and the driving circuit are omitted for the understanding of the connecting relationship between the treatment support instrument 58 and the manipulator portion 5.

As described above, by connecting an arbitrary treatment support instrument, such as an operating support instrument including a scalpel and a forcep, an inspection support instrument and a sensor, to the manipulator portion of the medical manipulator of the present invention, it is possible to provide further advanced functions to the medical manipulator of the present invention.

Next, the connection condition between the medical manipulator of the present invention and the subject or the medical imaging device will be described with reference to FIGS. 9A to 9D. FIGS. 9A and 9B are schematic arrangement views, seen from a width direction and a longitudinal direction, respectively, of a medical image system in which the medical manipulator of the present invention is joined to a movable bed which includes a bed base 60 and a bed 59. Although not illustrated in FIGS. 9A and 9B, the subject 40 may be restrained on the bed using a positioning member, such as a belt and a shock absorbing material. Therefore, it is possible to consider that, by fixing the support unit 4 with respect to the bed, the support unit 4 is substantially positioned with respect to the subject 40. The support unit 4 is configured to support the driving unit 6 and the manipulator portion 5. Desirably, the support unit 4 is rigid so as to support the driving unit 6 and the manipulator portion 5 stably during operation of the medical manipulator. From the viewpoint of the positioning degree of freedom of the driving unit 6 with respect to the support unit 4, the support unit 4 may desirably include an adjustment mechanism having a degree of freedom for the adjustment of rotation, and the position and the direction of a straight line and a curve, and the like. FIGS. 9A and 9B illustrates a form provided with an adjustment mechanism with which the height direction position, the horizontal position, an angle of direction and an elevation angle may be adjusted. The above-described adjustment mechanism may include a predetermined driving source and may perform positioning by remote control. FIGS. 9C and 9D are schematic arrangement views in which the medical manipulator of the present invention is applied to the medical imaging device which includes the movable bed illustrated in FIGS. 9A and 9B. In the medical manipulator of the present invention, the work distance and the dimension are controlled so as not to affect imaging by the medical imaging device. In FIGS. 9C and 9D, the medical manipulator of the present invention is connected to the bed 59 so as to be disposed within a cylindrical measurement unit 65 of the MRI device 64.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-135454, filed Jun. 15, 2012, which is hereby incorporated by reference herein in its entirety.

1. A medical manipulator comprising:
a driving unit configured to at least include a vibration-type actuator;
a manipulator portion configured to at least include an inserting portion to be inserted in a living body and configured to be connected to the driving unit and be moved when driven by the driving unit;
a support unit configured to support the driving unit and the manipulator portion;
a driving circuit configured to be connected to the vibration-type actuator and output a driving signal which drives a vibration-type actuator to the vibration-type actuator; and
a stress compensation unit configured to reduce stress produced in the inserting portion due to a motion of the living body.

2. The medical manipulator according to claim 1, wherein the stress compensation unit includes:
a displacement detecting unit configured to detect positional displacement of the inserting portion caused by the living body; and
a compensation calculating unit configured to be connected to the displacement detecting unit and generate a compensation signal as a calculation result on the basis of displacement information output from the displacement detecting unit.

3. The medical manipulator according to claim 2, wherein the displacement detecting unit includes:
an in-body image obtaining unit configured to obtain image information output from a medical imaging device; and
a displacement calculator configured to perform image extraction of displacement of at least either one of the inserting portion or the living body in accordance with the image information and convert the displacement into the displacement information.

4. The medical manipulator according to claim 3, wherein the displacement calculator converts the displacement into the displacement information in accordance with a difference between displacement of the inserting portion and displacement of the living body.

5. The medical manipulator according to claim 1, wherein the stress compensation unit includes:
a stress detecting unit configured to detect positional displacement which the inserting portion receives by the living body; and
a compensation calculating unit configured to be connected to the stress detecting unit and generate a compensation signal as a calculation result on the basis of stress information output from the stress detecting unit.

6. The medical manipulator according to claim 5, wherein the stress detecting unit includes:
a force sensor configured to be fixed to at least any of the manipulator portion, the driving unit and the support unit and detect external force applied to at least any of the manipulator portion, the driving unit and the support unit;
a stress calculator configured to convert external force information output from the force sensor into the stress information.

7. The medical manipulator according to claim 5, wherein the stress detecting unit includes:
a plurality of force sensors configured to be fixed to at least any of the manipulator portion, the driving unit and the support unit, to detect external force applied to at least any of the manipulator portion, the driving unit and the support unit, and to be fixed at different positions from one another; and
a stress calculator configured to convert external force into the stress information in accordance with the plurality of pieces of external force information which are output from at least two of the plurality of force sensors.

8. The medical manipulator according to claim 5, wherein the stress detecting unit includes:
an in-body image obtaining unit configured to obtain image information output from a medical imaging device; and
a displacement stress conversion calculator configured to convert displacement information of at least any one of the inserting portion or the living body extracted in accordance with the image information into the stress information.

9. The medical manipulator according to claim 8, wherein the displacement stress conversion calculator converts the displacement information into the stress information in accordance with a difference of the inserting portion and displacement of the living body.

10. The medical manipulator according to claim 2, wherein:
the driving circuit is connected to a higher-order controller which outputs a control command for instructing control of the vibration-type actuator to the driving circuit; and
the compensation calculating unit is connected to a path from the higher-order controller to the vibration-type actuator.

11. The medical manipulator according to claim 10, wherein the compensation calculating unit is connected to a path from the higher-order controller to the driving circuit so as to reduce stress caused in the inserting portion due to a motion of the living body.

12. The medical manipulator according to claim 1, wherein the manipulator portion includes a treatment support instrument.

13. The medical manipulator according to claim 3, wherein the medical imaging device is at least one of a nuclear magnetic resonance imaging device, an ultrasonic imaging device, a radiation imaging device and a photo-acoustic wave imaging device.

14. A medical manipulator according to claim 1, wherein:
the medical manipulator is constituted by the driving circuit and the vibration-type actuator; and
the medical manipulator further comprising a plurality of driving elements which cause the manipulator portion to move in different directions.

15. The medical manipulator according to claim 14, wherein the medical manipulator includes a plurality of stress compensation units; and
at least two of the plurality of stress compensation units are connected to at least two of the plurality of driving elements which cause the manipulator portion to move in different directions.

16. The medical manipulator according to claim 1, wherein the stress compensation unit is connected to a compensation instruction unit configured to output a compensation instruction to the stress compensation unit when it is detected that the manipulator portion has arrived at a specific site of the living body, and to perform a compensation operation in accordance with the compensation instruction.

17. A medical imaging system comprising:
the medical manipulator according to claim 16;
a medical imaging device configured to perform imaging of in-body information of the subject,
wherein the compensation instruction unit is included in medical imaging device.

18. A medical imaging system, comprising:
the medical manipulator according to claim 3; and
a medical imaging device configured to perform imaging of in-body information of the subject,
wherein the higher-order controller is included in the medical imaging device.

19. The medical imaging system according to claim 17, wherein the medical imaging device is at least any of a nuclear magnetic resonance imaging device, an ultrasonic imaging device, a radiation imaging device and a photo-acoustic wave imaging device.
